(19) United States
(12) Patent Application Publication
(10) Pub. No.: US 2016/0325075 A1
(43) Pub. Date: Nov. 10, 2016

(54) MATERIALS, DEVICES, SYSTEMS, AND METHODS FOR IDENTIFYING FEMALE URETHRA

(71) Applicant: C.R. BAR, INC., Murry Hill, NJ (US)

(72) Inventors: Henri Madigan, Conyers, GA (US); David Matthew Goldman, Decatur, GA (US); Emily Lewis, Longanville, GA (US); Richard Reineke, Atlanta, GA (US); Shannon Russell, Conyers, GA (US); Fung Bor Chen, Covington, GA (US); Beatrice Wan, Roswell, GA (US); Gregory Mann, Covington, GA (US); Joshua Daniel Ziadie, Atlanta, GA (US)

(21) Appl. No.: 15/111,689

(22) PCT Filed: Jan. 21, 2015
(86) PCT No.: PCT/US2015/012297

§ 371 Date: Jul. 14, 2016

Related U.S. Application Data
(60) Provisional application No. 61/935,249, filed on Feb. 3, 2014.

(51) Int. Cl. A61M 25/01; A01N 59/12; A61K 49/00; A61M 25/00; A61B 42/10

(52) U.S. Cl. CPC A61M 25/01; A61M 25/0017; A61B 42/10; A61K 49/006; A01N 59/12; A61M 2205/0227; A61M 2205/584; A61M 2210/1092; A61M 2210/1475

(57) ABSTRACT

Embodiments of the invention include materials, devices, systems, and methods for identifying female urethra and facilitating catheterization. For instance, materials, devices, systems, and methods described herein may facilitate marking of female urethra, which may lead to increase in insertion accuracy and may reduce time for completing the catheterization procedure.

### BACKGROUND

Catheterization is a relatively common procedure in medical practice. For example, inserting a catheter into a patient's bladder may allow urine to drain freely therefrom. In some instances, a catheter also may facilitate injection of liquids into the bladder (e.g., to treat bladder conditions). Typically, catheterization is performed by inserting a catheter through a patient's urethra into the bladder.

Variance in layouts of female genitalia, which may vary due to age, obesity, childbirth, other reasons, or combinations thereof commonly present a challenge to identifying the patient's urethra. A typical visualization method for identifying the female urethra involves swabbing the area with iodine and looking for a momentary pooling of iodine at a meatus opening of the female urethra. Identifying the urethral opening may be difficult with typical visualization methods. Moreover, difficulty with identifying the urethra may result in a prolonged procedure and patient discomfort.

### SUMMARY

Embodiments of the invention include materials, devices, systems, and methods for identifying a female urethra and facilitating catheterization. For instance, such materials and/or devices may reduce the time typically required for a catheterization procedure. More specifically, the materials, devices, systems, and methods described herein may facilitate marking of a female urethra, which may lead to an increase in insertion accuracy. Furthermore, identification of the urethra may reduce time required for completing the catheterization procedure, particularly on female patients.

An embodiment involves a method of inserting a catheter into a urethral opening of a female patient. The method includes locating the urethral opening. The method additionally includes, while the identifiable marker is positioned on and/or near the urethral opening, placing an identifiable marker on and/or near the urethral opening. The method further includes inserting the catheter into the urethral opening at least in part based on the position of the identifiable marker.

Embodiments also include a glove configured for insertion of a catheter into a urethral opening of a female patient. The glove includes a shell defining a shape of the glove including a palm and multiple fingers. The shell includes a flexible material. The glove also includes a guide extending along a finger of the multiple fingers of the shell. At least a portion of the guide is attached to or integrated with the finger. Material of the guide defines a channel extending from a proximal end toward a distal end of the guide. The channel is sized and configured to movably accept a catheter therein.

Additional or alternative embodiments include a sterilizing solution for sterilizing catheterization site and identifying a urethral opening of a female patient. The sterilizing solution includes an iodine solution and a reactive material mixed configured to react with one or more of urine or urethral mucus. The reaction of a portion of the reactive material with urine and/or urethral mucus produces a color change in the reacted portion of the reactive material.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. For better understanding, the like elements have been designated by like reference numbers throughout the various accompanying figures. Understanding that these drawings depict only typical embodiments of the invention and are not therefore to be considered to be limiting of its scope, the invention will be described and explained with additional specificity and detail through the use of the accompanying drawings in which:

FIG. 1 is a flow diagram of a catheterization method according to an embodiment of the invention;

FIG. 2 is a schematic cross-sectional view of a female urethra and a marker applied thereon to provide a target for catheterization according to an embodiment;

FIG. 3 is a flow diagram of a catheterization method according to an embodiment of the invention;

FIG. 4 is a cross-sectional view of a marker according to an embodiment;

FIG. 5 is a top view of a marker according to another embodiment;

FIG. 6 is an isometric view of a glove for a catheterization procedure according to an embodiment;

FIG. 7A illustrates an act of a catheterization procedure according to an embodiment;

FIG. 7B illustrates another act of the catheterization procedure according to an embodiment;

FIG. 8 is a top view of a glove for a catheterization procedure according to another embodiment;

FIG. 9A illustrates an act of a catheterization procedure according to an embodiment;

FIG. 9B illustrates another act of the catheterization procedure according to an embodiment;

FIG. 10 illustrates yet another act of the catheterization procedure according to an embodiment; and

FIG. 11 is a flow diagram of a method for marking a catheterization site according to another embodiment of the invention.

### DETAILED DESCRIPTION

Embodiments of the invention include materials, devices, systems, and methods for identifying a female urethra and facilitating catheterization. For instance, such materials and/or devices may reduce the time typically required for a catheterization procedure. In some examples, the materials, devices, systems, and methods described herein may facilitate marking of a female urethra, which may lead to an increase in insertion accuracy. Furthermore, identification of the urethra may reduce time for completing the catheterization procedure, particularly on female patients.

An embodiment includes a marker or marking material that may be placed on and/or near urethral opening, and which may be visible to a user or practitioner performing catheterization. Particularly, the marker may be affixed or attached to the patient's anatomy at and/or near the urethral opening and may remain in place and/or at least substantially in the same position during catheterization. As such, the marker may provide a visible target for aligning and/or inserting the catheter and may facilitate catheterization of the patient.

In some embodiments, the marker may have an easily identifiably and/or visible color, texture, luminescence, or combinations thereof. For example, a marker that includes white marking material may contrast against a dark background, such as the patient's anatomy, thereby providing a visible target on and/or near the patient's urethra. Also, in one or more embodiments, the patient's anatomy (e.g., on and/or near the urethral opening) may be covered or coated with a base material or substance that may provide a background, which may increase contrast or visibility of the marker. For instance, the base substance may include an iodine solution that may have a dark color (e.g., brown), and the marker may have a light color (e.g., white). Accordingly, the light-colored marker may stand out or contrast against the background color or base coat and may be easily visible or identifiable against the dark-colored background color of the base substance or base coat. Additionally, the base substance may sterilize the patient's anatomy, thereby providing a sterilized environment at and/or near the catheterization site.

Additionally or alternatively, the marker may include a marking material that may react with substances on and/or near a patient's anatomical features (e.g., body fluids). In at least one embodiment, the marking material may react with substances present on and/or near a urethral opening, such as urine. In particular, reaction of the marking material with the substance on the patient's anatomy may change the color of the marking material (e.g., in a manner that increases contrast thereof relative to the anatomical features marked thereby).

In one or more embodiments, a marking material may react with a substance to produce florescent molecules, which may glow or emit radiation (e.g., when exposed to light). Moreover, in some instances, reaction between the marking material and the substance may produce luminescence (e.g., chemiluminescence). In any event, the marking material may provide a visible target that may remain visible and in a position on and/or near the urethral opening during the catheterization procedure.

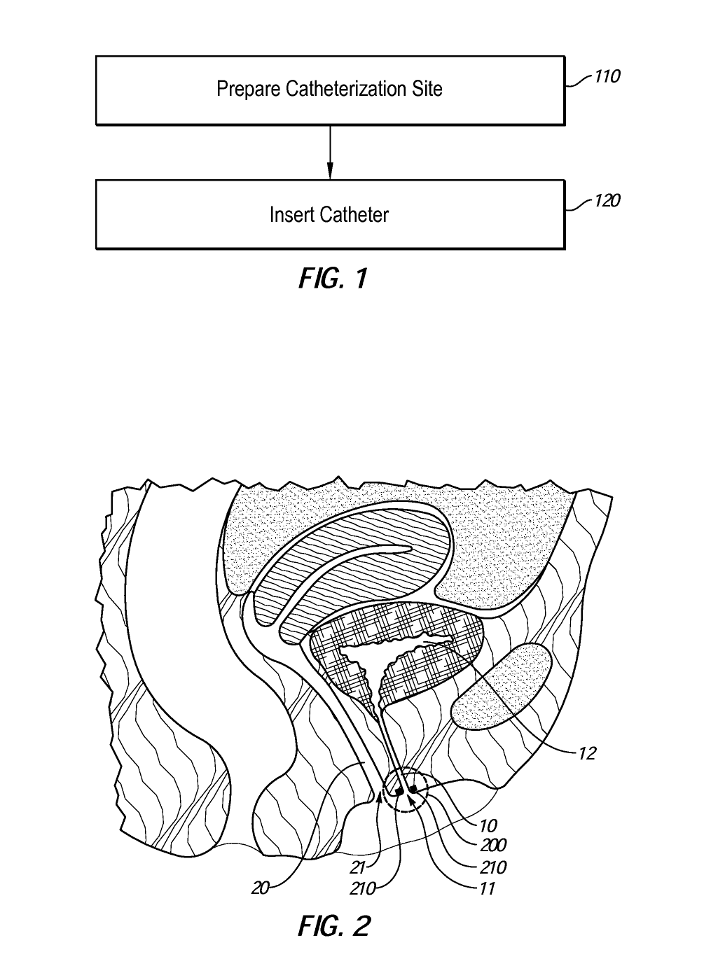

FIG. 1 illustrates a catheterization procedure according to an embodiment. For example, as shown in FIG. 1, the catheterization procedure may include an act 110 of preparing a catheterization site. Additionally, the catheterization procedure may include an act 120 of inserting the catheter (e.g., inserting the catheter into patient's urethral opening). In some embodiments, the catheterization procedure may involve marking the catheterization site or target. In particular, in some embodiments, the act 110 may involve cleaning and/or sterilizing the catheterization site and marking the urethral opening. Furthermore, it should be appreciated that, in some instances, after insertion, the catheter may be advanced along the patient's urethra to a suitable location (e.g., into the patient's bladder).

FIG. 2 is a diagrammatic illustration of a female anatomy at and near a catheterization site 200 according to an embodiment. More specifically, FIG. 2 illustrates a urethra 10 and corresponding urethral opening 11 near the catheterization site 200. In addition, FIG. 2 illustrates a vaginal canal 20 and a corresponding vaginal opening 21 located near the urethral opening 11. Generally, a catheter may be inserted through the urethral opening 11 and into the urethra 10. Furthermore, as noted above, the catheter may be advanced to any suitable location (e.g., into patient's bladder 12).

As illustrated in FIG. 2, the catheterization site may be marked by placing a marker on and/or near the catheterization site, to facilitation identification thereof. More specifically, in an embodiment, a marker 210 may be placed at and/or near the urethral opening 11. In some examples, the marker 210 may be placed between the urethral opening 11 and the vaginal opening 21. Hence, for instance, the marker 210 may assist the practitioner with identifying the urethral opening 11 and/or distinguishing the urethral opening 11 from the vaginal opening 21. Additionally or alternatively, the marker 210 may be placed above the urethral opening 11 and away from the vaginal opening 21 and/or near or within the catheterization site 200. In any event, the marker 210 may facilitate identification and/or may provide a target for the urethral opening 11, thereby simplifying insertion of a catheter into the urethral opening 11.

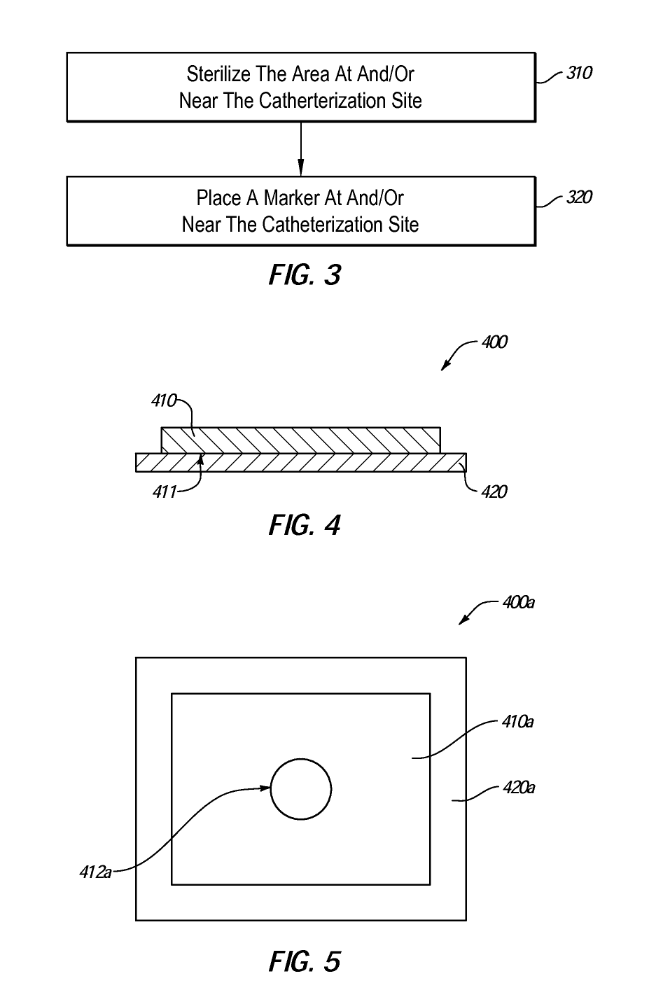

At least one embodiment, as illustrated in FIG. 3, the catheterization procedure may include an act 310 of sterilizing the area at and/or near the catheterization site. For example, the method may include applying an antiseptic or antibacterial substance in the area, such as a sterilizing solution. Generally, the antibacterial substance may vary from one embodiment to the next. In some embodiments, the antibacterial substance may have a dark color. For instance, antibacterial substance may be a sterilizing solution of an iodine, Povidone-iodine, or combinations thereof which may be used to sterilize the area.

In some embodiments, the sterilizing solution may have a dark color. Accordingly, such solution also may form a background or base coat for a marker, which may be applied at and/or near the catheterization site, as further described below. The sterilizing solution may naturally have a dark color and/or a coloring may be added to an otherwise light or clear sterilizing solution to achieve a desired color. For example, a food coloring may be added to the sterilizing solution to achieve the desired color. In some instances, as described above, the color of the sterilizing solution may be substantially dark, such as brown, black, or the like. Alternatively, the color of the sterilizing solution may be light, such as tan, white, or the like. In any event, applying the sterilizing solution to the area at and/or near the catheterization site may sterilize the site as well as provide a background or base coat of a suitable color to contrast against the marker (e.g., such that the marker is easily visible against the background color, such as the base coat color).

The method further includes an act 320 of placing a marker at and/or near the catheterization site. The marker may have a dark, light, or otherwise visible color, which may be contrasted against the background. It should be appreciated that the background color may be the color of the patient's anatomy, color of the base coat (e.g., described above), or combination thereof. The marker may include any number of suitable materials and/or colors, which may vary from one embodiment to the next. Generally, the color of the marker may be chosen in a manner that provides a visible target at the catheterization site. For instance, if the background color is dark (e.g., after applying a base coat, such as a Povidone-iodine solution), the marker may have a light color, such as white.

In at least one embodiment, the marker may include a marker material that is in a powder form or otherwise granular. Grain size of the powder may vary from one embodiment to the next. Furthermore, in some embodiments, the powder may have a light color. For instance, the power may have a white color, such as talcum powder. In any event, the powder may be visible against a dark background that a sterilization solution may provide on and/or near the urethral opening.

In some embodiments, the powder may adhere to the sterilization solution and/or to the anatomy. For instance, sterilization solution may be a liquid that may form a film or a coating on the patient's anatomy. The marker may adhere to such film or coating. In any case, however, the marker may attach to the anatomy at and/or near the catheterization site and may remain there sufficiently long to facilitate insertion of the catheter. For instance, the marker may remain attached and/or visible for a period of time in one or more of the following ranges: between about 0.5 minutes and about 2 minutes; between about 1 minute and about 4 minutes; and between about 3 minutes and 10 minutes. In some embodiments, the marker may remain attached and/or visible for a period that is less than 0.5 minutes or greater than 10 minutes.

In some embodiments, to facilitate attachment, an adhesive marker may include an adhesive that may secure the marker at a suitable location. FIG. 4 illustrates an adhesive marker 400 according to an embodiment. In at least one embodiment, the adhesive marker 400 may include a marking panel 410, such as a thin plate, a fabric, or other suitable backing material with an adhesive (e.g., a pressure sensitive adhesive) on the attachment side 411 thereof.

In some examples, a protector 420 may be removably attached on the attachment side 411 of the adhesive marker 400 and may prevent unintentional or accidental attachment of the adhesive marker 400 (e.g., the protector 420 may cover or conceal the adhesive on the attachment side 411 of the adhesive marker 400). In at least one embodiment, the adhesive marker 400 may be pressed against the anatomy at the suitable location to activate the adhesive, thereby attaching the marker to the anatomy.

As described above, the adhesive marker 400 may have a suitable color that may be visible during catheterization. For example, the backing material may have a white color and may be visible against a dark background color. Moreover, embodiments also may include the adhesive marker 400 that may exhibit luminescence. Generally, the adhesive marker 400 may include any suitable light emission mechanism, such as fluorescent emission and phosphorescent emission. In some instances, light emitted by the adhesive marker 400 may be visible in the dark. Alternatively or additionally, light emitted from the adhesive marker 400 may be visible when the marker is exposed to a particular wavelength of light (e.g., UV light). It should be also appreciated that any marker described herein may exhibit any number of suitable light emission properties, as described herein.

Furthermore, the adhesive marker 400 may be detached and/or removed from the site after completing catheterization. Hence, in some embodiments, the adhesive marker 400 may remain attached at and/or near the site of catheterization for any suitable amount of time, until manually removed from the site. Additionally or alternatively, the pressure sensitive adhesive of the adhesive marker 400 may be configured to degrade in a manner that reduces adhesive strength thereof with time. Hence, in some embodiments, the adhesive marker 400 may detach after the passing of a predetermined amount of time.

As shown in FIG. 5, according to at least one embodiment, a marker 400a (e.g., an adhesive marker) may include a hole or an opening 412a in a marking panel 410a thereof. In one or more embodiments, except as otherwise described herein, the marker 400a and its elements and components may be similar to or the same as the adhesive marker 400 (FIG. 4). For example, the marker 400a may include adhesive on an attachment side thereon. In an embodiment, the opening 412a may demarcate the urethral opening, such that after attaching the adhesive marker 400a at and/or near the catheterization site, the marking panel 410a, which defines the opening 412a, may surround the urethral opening.

Generally, the marker 400a may have any suitable shape that may vary from one embodiment to the next. In the illustrated embodiment, the marker 400a has an approximately rectangular shape. In some embodiments, the marker may have an oval shape, a circular shape, an irregular shape, etc. Furthermore, in an embodiment, the opening 412a in the marker may be approximately concentrically aligned with the urethral opening. As such, the adhesive marker may indicate a target location by establishing a visible perimeter about the urethral opening.

Similar to the adhesive marker 400 (FIG. 4), for the marker 400a that includes adhesive on the attachment side thereof, a protector 420 may be attached to the attachment side of the marker 400a, such as to cover the adhesive thereon and/or prevent unintentional or accidental adhesion of the marker 400a. It should be appreciated that the periphery of the protector 420a may have any suitable shape and/or size that may facilitate removal thereof from the marking panel 410a (e.g., before attaching the marker 400a at and/or near the catheterization site).

As described below in more detail, in some embodiments, the marker (e.g., the adhesive marker 400 and/or marker 400a (FIGS. 4, 5)) may be preliminarily attached to a glove (e.g., attached with an adhesive to a finger on the glove, such as the middle finger). After securing the adhesive marker at the suitable location, the adhesive may detach from the glove. In some examples, the adhesive securing the adhesive marker to the patient's anatomy may be stronger than the adhesive or other mechanism (e.g., mechanical fastening, such as a portion of the glove's finger fitted through the hole) securing the adhesive marker to the glove. In some instances, the adhesive marker may be manually removed from the glove's finger after attachment at the anatomical location.

Embodiments also may include markers that incorporate adhesive materials and/or materials that may adhere at the suitable location of patient's anatomy. For instance, a gel marker may include a water soluble polymer, which may have a visible color or produce luminescence under operating conditions. In some embodiments, the water soluble polymer may include Aloe vera gel, alcohol gel, polyethylene glycol, polyvinyl alcohol, polyacrylic acid, among others. In some embodiments, a food coloring may be added to the water soluble polymer of the gel marker to produce a suitable color.

The gel marker may be smeared or otherwise applied at the suitable site, such as applied at and/or near the urethral opening. In at least one embodiment, the gel marker also may disinfect the area at and/or near the urethral opening. Alternatively or additionally, the gel marker may provide lubrication at the urethral opening, which may facilitate insertion of the catheter into the opening. In addition, because the gel marker may be water soluble, any portion of the gel marker that may enter the patient's urethral opening and/or bladder may dissolve and flush out with urine.

In one or more embodiments, the gel marker may be placed above or near the target. For instance, the gel marker may form a dot or a line above the urethral opening (e.g., away from the vaginal opening). Accordingly, a practitioner performing the catheterization procedure may visualize the location of the urethral opening by correlating the location of the opening with the location of the marker.

In one or more embodiments, the gel marker may be applied directly onto the urethral opening and provide sufficient visual identification thereof. Furthermore, while in some instances the gel marker may include a static color or may exhibit luminescence under operating conditions, in at least one embodiment, material of the gel marker may include a reactive material that may react with substances and/or material at and/or near the catheterization site to produce a color change or luminescence.

In some embodiments, a batch or glob of gel may form the gel marker. Furthermore, the gel marker may include a backing (e.g., removable backing) that may facilitate handling thereof and/or may prevent unintentional or accidental attachment of the gel marker. In some embodiments, the gel marker may be stored (e.g., before application thereof) in a bag or similar packaging, which may prevent or minimize contamination of the gel marker and/or unintentional or accidental attachment thereof. In one or more embodiments, the gel marker may be substantially sterile, which may prevent contamination of the catheterization site.

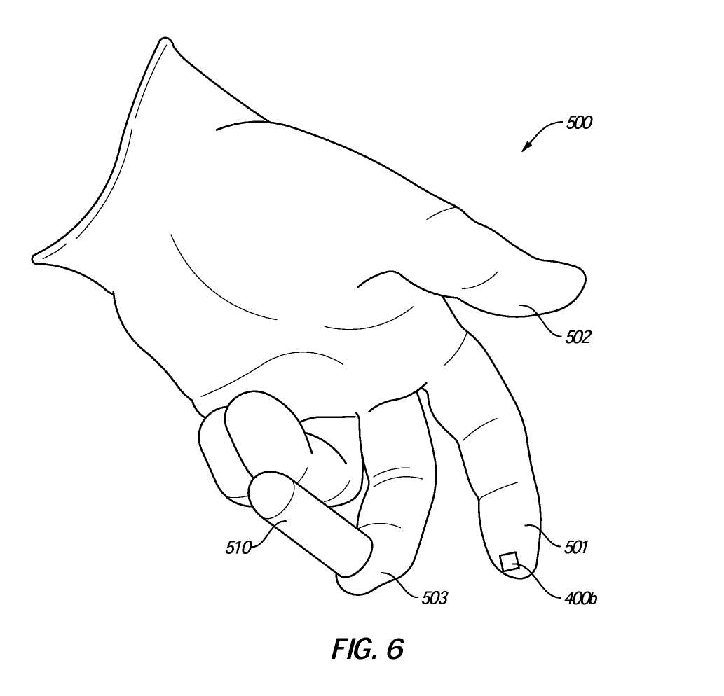

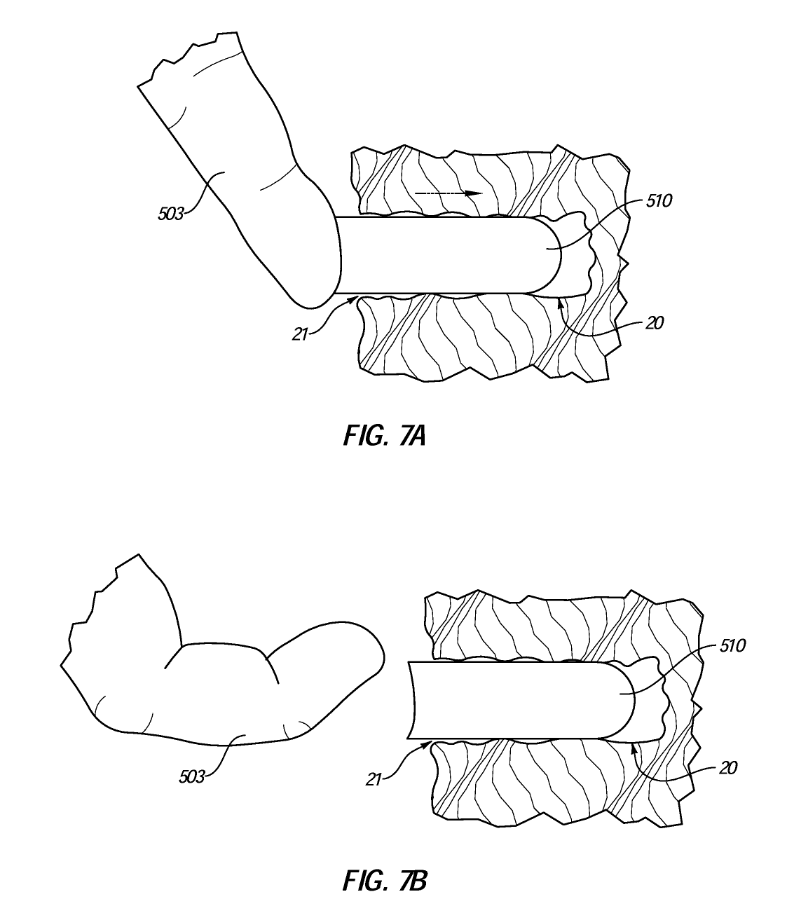

As noted above, in some embodiments, the marker may be attached or temporarily secured to a glove, and a practitioner may apply and/or attach the marker at and/or near the catheterization site, also detaching the marker from the glove. FIG. 6 illustrates a glove 500 that has a marker 400b attached thereto, according to an embodiment. Generally, the glove 500 includes and/or is defined by a shell (e.g., a flexible shell, such as including latex or similar flexible material) that forms the general shape of the glove 500.

In addition, as shown in FIG. 6 and described below in more detail, the shell of the glove 500 may include a palm and multiple fingers, such that the shell of the glove 500 approximates the shape of a human hand (e.g., having five fingers). In other words, in some embodiments, a practitioner or user may don the glove 500 on either the right or the left hand. Hence, in some examples, the glove 500 may be configured for a specific hand; in additional or alternative embodiments, the glove 500 may be configured to be worn on the right and the left hand in a similar manner. The glove 500 may have any suitable size, which may vary from one example to another. Furthermore, the flexible material of the glove 500 may permit the glove 500 to stretch to a suitable size to accommodate hand sizes larger than glove 500. Moreover, in some embodiments, the glove 500 may be substantially sterile.

Generally, the glove 500 may have any number of suitable markers attached or secured to any number of suitable portions thereof. The marker 400b may be similar to or the same as any marker described herein. For example, the gel marker (described above), adhesive marker 400, marker 400a (FIGS. 4-5), combinations thereof, etc., may be attached to the glove 500.

As described above, the marker 400b may be attached to the glove 500 with adhesive(s), with an interference fit between a portion of the glove 500 and a portion of the marker 400 (e.g., the marker 400b may include an opening an a portion of the glove 500 may be secured within the opening of the marker 400b). In any event, in some embodiments, the marker 400b may be removably attached or secured to the glove 500, such that the marker 400b may be detached from the glove 500 after application of the marker 400b at and/or near the catheterization site.

Generally, the marker 400b may be attached at any suitable location on the glove 500. For example, the marker 400b may be attached on the index finger 501 of the glove 500 (e.g., at the tip of the index finger 501). Under some operating conditions, a practitioner may wear the glove 500 and may attach the marker 400b at and/or near the catheterization site by pressing the index finger, which includes the marker 400b, at a suitable location, thereby attaching the marker 400b at such location, and detaching the marker 400b from the glove 500.

Additionally or alternatively, the marker 400b may be attached at one or more other locations on the glove (e.g., on and/or near the thumb 502 of the glove 500). Moreover, as noted above, the glove 500 may form or define the protector or backing for the marker 400b (e.g., for an adhesive marker). Hence, for instance, the practitioner wearing the glove 500 may remove the marker 400b from the glove 500 and attach the marker 400b at a suitable location at and/or near the catheterization site.

One or more embodiments include a positional blocker, which may be placed or positioned near the catheterization site in a manner that prevents or limits advancement of the catheter to locations outside of the urethral opening. For example, a positional blocker 510 may be inserted into the vaginal opening, such that the positional blocker 510 remains in the vaginal canal and prevents or limits advancement of the catheter into the vaginal opening, thereby facilitating insertion of the catheter into the urethral opening.

In an embodiment, the positional blocker 510 may be attached to the glove 500. For example, a practitioner wearing the glove 500 may easily insert the positional blocker 510 through the patient's vaginal opening and into the vaginal canal. In some embodiments, the positional blocker 510 may be attached to the middle finger 503 of the glove 500 (e.g., at and/or near the tip of the middle finger 503). Furthermore, in at least one embodiment, the positional blocker 510 may be attached to extend approximately perpendicularly relative to the middle finger 503 of the glove 500. In other words, when the practitioner wears the glove 500, unfolding the practitioner's hand and facing the hand palm upward, may positional blocker 510 to extend upward from the middle finger 503 (e.g., such that the positional blocker is oriented approximately perpendicularly relative to the middle finger 503).

It should be appreciated that the positional blocker 510 may be attached to any other suitable portion of the glove 500 (e.g., on any finger of the glove 500). Moreover, the positional blocker 510 may be attached in a manner that orients the positional blocker 510 relative to the glove 500 (e.g., relative to the finger of the glove) or relative to the practitioner's hand wearing the glove 500. For example, the positional blocker 510 may be oriented relative to the glove 500 or any portion thereof at any suitable angle (e.g., at an angle that is less or greater than 90°, such as at 120°, at 45°, at 30°, etc.). In any event, the positional blocker 510 may be attached at a suitable location of the glove 500 and oriented in a suitable manner, such as to facilitate insertion thereof into the vaginal opening of the patient.

FIGS. 7A-7B illustrate a catheter insertion sequence according to an embodiment. For example, as shown in FIG. 7A, the positional blocker 510 may be inserted into the vaginal opening 21 and may enter the vaginal canal 20. Moreover, in some embodiments, the positional blocker 510 may be inserted into the vaginal canal 20 to a suitable distance, such that the positional blocker 510 may be secured in the vaginal canal 20 (e.g., for the duration of the catheterization procedure) and/or a portion of the positional blocker 510 may protrude out of the vaginal opening 21. Hence, generally, the positional blocker 510 may have any suitable size (e.g., peripheral size, such as diameter) and length to facilitate securing the positional blocker 510 in the vaginal canal 20.

Generally, after inserting the positional blocker 510 into the vaginal canal 20, the practitioner may detach the positional blocker 510 from the glove 500 in any number of suitable ways. For example, as shown in FIG. 7B, the practitioner may bend the finger to which the positional blocker 510 is attached (e.g., middle finger 503), thereby detaching the positional blocker 510 from the glove. Additionally or alternatively, the practitioner may pull the finger and/or hand away from the vaginal opening 21, which may detach the positional blocker 510 from the glove (e.g., from the finger 503 of the glove).

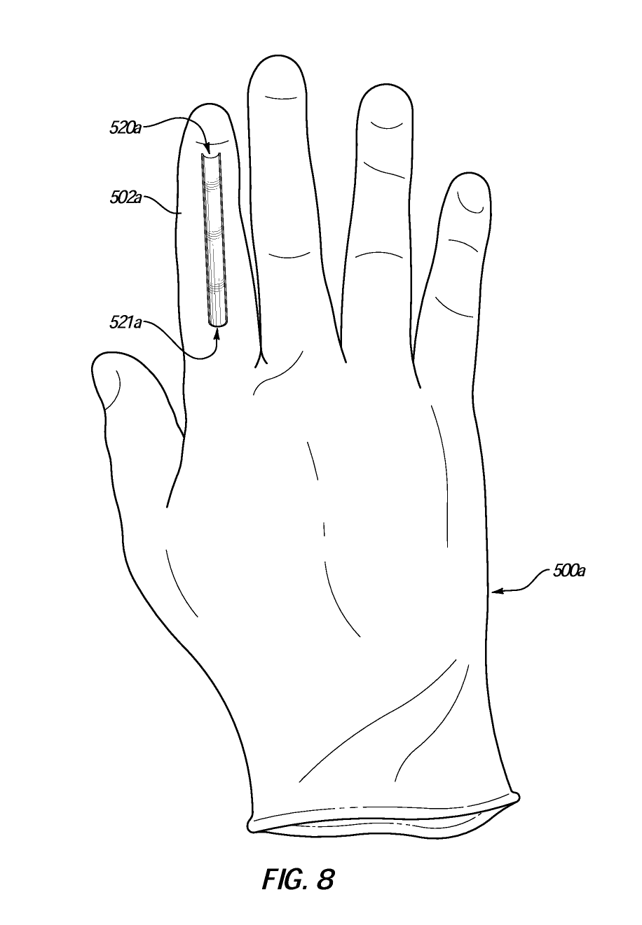

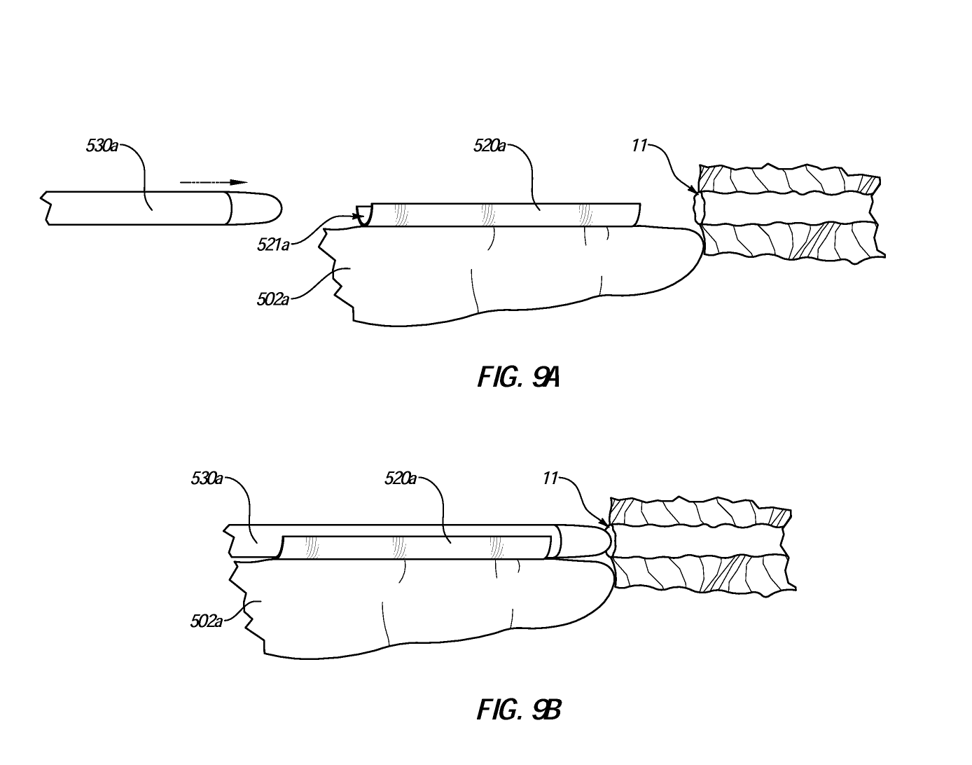

One or more embodiments may include one or more mechanisms for guiding the catheter in a manner that may improve or facilitate the catheterization procedure. Furthermore, such guiding mechanism(s) may be attached to or incorporated with the glove that may be worn by the practitioner during the catheterization procedure. FIG. 8 illustrates a glove 500a according to an embodiment, which includes a guide 520a. More specifically, for instance, the guide 520a may be configured to guide the catheter toward and/or into the urethral opening of the patient, thereby facilitating insertion of the catheter into the urethral canal of the patient.

For example, as described below in more detail, the practitioner may align the guide 520a with a marker positioned at and/or near the catheterization site and/or with the urethral opening and may guide the catheter along the guide 520a and into the urethral opening of the patient. For example, in some embodiments, the catheterization site may be marked prior to or after insertion of the positional blocker 510 using any of the methods disclosed herein for marking a catheterization site using any of the identifiable markers disclosed herein, such as described in conjunction with embodiments shown in FIGS. 1-6. In other embodiments, the catheterization site may be located using the so called “blink method” in which an area at and/or near the catheterization site is swabbed with iodine and the user looks for a momentary pooling of iodine at an opening in the urethra. Generally, the guide 520a may have any suitable shape, which may facilitate guiding or channeling the catheter along the guide 520a and toward the urethral opening. In an embodiment, the guide 520a includes a channel 521a (e.g., exterior walls or material of the guide 520a may define or form the channel 521a), which may be sized and configured to facilitate movement and/or sliding of the catheter along the guide 520a and toward the urethral opening of the patient.

In some embodiments, the channel 521a may be open at opposing ends of the guide 520a, such that the catheter may be inserted at a proximal end of the channel 521a and advanced toward and through a distal (opposite) end of the channel 521a. Additionally or alternatively, the channel 521a may be open at the top thereof, such that the catheter may be placed into the channel 521a (e.g., the channel 521a may be U-shaped, V-shaped, etc.). In any event, the catheter may be advanced along the guide 520a from the proximal end (closest to the practitioner) to the distal end (closest to the patient) and may be subsequently inserted into the patient's urethral opening.

As mentioned above, the guide 520a may be attached to and/or integrated with the glove 500a (e.g., all or a portion of the guide 520a may be integrated with the glove 500a). Moreover, the guide 520a may include any number of suitable materials, which may facilitate attachment and/or integration of the guide 520a with the glove 500a. In some embodiments, the guide 520a may include material and/or coating that may facilitate advancement of the catheter along the guide 520a (e.g., by reducing friction between the guide 520a and the catheter).

Generally, the guide 520a may be attached at any suitable location on the glove 500a. For example, the guide 520a may be attached to an index finger 502a of the glove 500a. In some embodiments, the guide 520a may be attached to an upward-facing side (e.g., side opposite to the palm side) of the glove 500a. In other words, when the practitioner wearing the glove 500a faces the palm of the gloved hand downward, the upward-facing side of the glove 500a and the guide 520a may face upward.

Furthermore, the guide 520a may have any suitable length and may extend along a portion of or the entire length of the index finger 502a of the glove 500a. The guide 520a may be attached along a portion or along the entire length thereof. For example, only a portion of the guide 520a may be attached or integrated with the glove 500a. Hence, for instance, one or more portions of the index finger 502a of the glove 500a may be movable relative to the guide 520a (e.g., at joints of practitioner's finger). In some examples, the practitioner may bend the finger inserted into the index finger 502a of the glove 500a to extend a portion of the guide 520a relative to the bend of the finger.

In any event, the practitioner may insert and/or guide the catheter along the guide 520a, which may facilitate insertion of the catheter into the urethral opening of the patient. FIGS. 9A-9B illustrate a catheter insertion sequence according to an embodiment. In particular, for example, as shown in FIG. 9A, the practitioner may position the index finger 502a of the glove relative to the urethral opening 11 of the patient, such that the channel 521a of the guide 520a may be aligned with the urethral opening 11 (e.g., such that catheter 530a may be advanced in the channel 521a toward and/or into the urethral opening 11). In any event, as shown in FIG. 9A, in at least one example, the catheter 530a may be advanced toward and/or into the proximal end of the guide 520a.

Generally, the size and/or shape of the channel 521a may vary from one embodiment to the next and may depend on the corresponding size and/or shape (e.g., cross-sectional shape) of the catheter 530a intended for insertion into the patient's urethral opening 11. For instance, a user may select a glove with the corresponding guide 520a that has a suitable sized channel 521a. In some embodiments, the glove and the catheter 530a may be packaged in a kit, such that the size and/or shape of the channel 521a suitably corresponds with the size and shape of the catheter 530a.

For instance, as shown in FIG. 9B, the catheter 530a may be advanced along the guide 520a from the proximal end to the distal end thereof (e.g., toward and/or to the tip of the index finger 502a and toward the urethral opening 11). In some examples, when the index finger 502a is in the extended or unbent position, the distal end of the guide 520a may be located proximally relative to the tip of the index finger 502a (e.g., the tip of the index finger 502a may extend beyond the distal end of the guide 520a). As described above, in some embodiments, only a portion of the guide 520a may be attached to or integrated with the index finger 502a. Hence, for instance, a portion of the index finger 502a may be moved relative to the guide 520a.

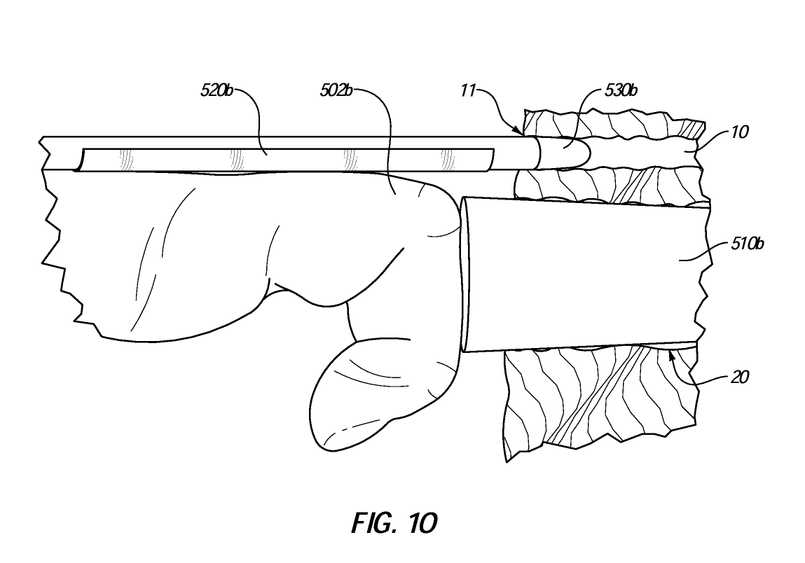

In some embodiments, as shown in FIG. 10, the practitioner may move or bend a distal portion of an index finger 502b away from a guide 520b. As mentioned above, in an embodiment, after bending the index finger 502b, the guide 520b may extend past the distal edge or face of the joint at the bend. In other words, the guide 520b may extend past the most distal portion of the bend index finger 502b. As such, the guide 520b may be positioned closer to the urethral opening 11 than the most distal face or portion of the bent index finger 502b.

Moreover, as described above, in some embodiments, the practitioner may insert a positional blocker 510b into the patient's vaginal canal 20. As shown in FIG. 10, for example, the positional blocker 510b (e.g., which may be similar to or the same as the positional blocker 510 (FIGS. 6-7B)) may prevent the practitioner from unintentionally inserting the catheter into the vaginal canal 20 instead of the urethral canal 10. Furthermore, the practitioner may bend the index finger 502b of the glove (e.g., by bending the finger inserted into the index finger 502b of the glove). For example, practitioner's bent finger (and the bend index finger 502b of the glove) may rest against a face of the positional blocker 510b (e.g., the positional blocker 510b may protrude out of the vaginal canal 20). In an embodiment, the guide 520b may protrude past the face of the positional blocker 510b, which extends out of the vaginal canal 20.

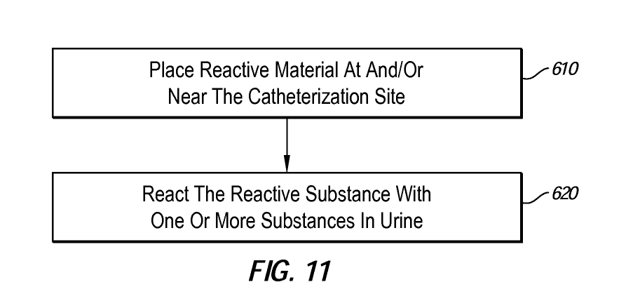

As mentioned above, catheterization procedure may involve placing a marker at and/or near the catheterization site. Moreover, in some embodiments, the marker may react with one or more substances at and/or near the catheterization site (e.g., with one or more biological fluids). For example, marking the catheterization site may include an act 610 of placing a reactive material at and/or near the catheterization site, as shown in FIG. 11. In one or more embodiments, the reactive material may be included or incorporated in the gel marker (described above) and may react with urine to produce a color change or luminescence. In some embodiments, the gel marker may react with urine in a manner that changes color of the gel marker and/or produced chemiluminescence.

In some instances, at least a trace amount of urine may be at and/or near the catheterization site (e.g., near the urethral opening). Accordingly, marking the catheterization site may include an act 620 of reacting the reactive substance (e.g., of the gel marker) with urine and/or one or more substances in the urine. In particular, for example, such reaction may produce a visual effect, such as color change or luminescence, may identify the location of the urethral opening.

The marker (e.g., gel marker, adhesive marker, etc.) may include any suitable chemical or material that may react with urine to produce a color change or luminescence. In addition, the material of the marker, including the reacting material, may be safe for application in, at, and/or near the target site and, in some instances, may be biodegradable. Suitable reactive materials include sodium nitroprusside, which may react with ketones, such as acetone, in urine to produce a purple color. Additionally or alternatively, reactive material may include alkaline picrates, which may react with creatinine in the urine to produce a reddish-orange color. Furthermore, the reactive material may include copper ions, which may react with urinary glucose to form brick-red colored precipitate. The reactive material also may include glucose oxidase enzyme, which may react with glucose in urine to produce a brown color. It should be appreciated that the marker may include any number of suitable additional or alternative reactive materials and combinations thereof, which may react with materials and/or substances in the urine to produce a change in color and/or luminescence.

Moreover, in an embodiment, the reactive materials may react to or with the mucosa of the urethral opening. In particular, reaction of the reactive materials and materials of the mucosa may produce a change in color, which may facilitate insertion of the catheter into the urethral opening. In any event, the reactive material may react with one or more substances or materials located at the urethral opening to produce a target-identifying color change.

Any of the markers described herein may include such reactive materials that may produce a color change in a reaction with materials and/or substances in urine (e.g., a portion of the reactive material that reacts with the materials or substances in urine may change color, while the remaining or unreacted portion of the reactive material may have an unchanged or original color). In an embodiment, the reactive material may be included or incorporated in the solution. For example, the Povidone-iodine solution may include sodium nitroprusside. Accordingly, after sterilizing the catheterization site at and/or near the urethral opening, the sodium nitroprusside may react with ketones in the urine, traces of which may present at and/or near the urethral opening. More specifically, reaction of the sodium nitroprusside with the urine traces may produce a purple color, which may be visible against an otherwise brown background provided by the Povidone-iodine portion that did not react with ketones. As such, the reaction may produce a marker that may facilitate accurate and quick insertion of the catheter into the urethral opening.

Furthermore, embodiments may involve illuminating the catheterization site with ultraviolet (“UV”) light. In particular, as mentioned above, the catheterization site may include traces of urine at and/or near the urethral opening. At least in some instances, urine may include fluorescent molecules, which may glow or emit light when exposed to UV light. Thus, exposing the catheterization site to UV light may facilitate identifying the urethral opening and inserting a catheter therein.

In some embodiments, a hand-held device may be used to provide UV illumination of the catheterization site. For example, a portable device, such as a pen-shaped device, may provide suitable illumination of the catheterization site. The device also may be configured to be reusable. Hence, in some embodiments, the device may include materials that may withstand sterilization treatments.

In yet further embodiments, placing a marker at the catheterization site may include applying a more viscous sterilization solution to the catheterization site. For example, viscosity of the Povidone-iodine may be increased to thicken the typical Povidone-iodine solution. Specifically, in one embodiment, the area at and/or near the urethral opening may be wiped with a viscous Povidone-iodine solution. Subsequently, the practitioner may observe the viscous Povidone-iodine solution run down into the urethral opening, thereby creating a blinking effect. While with a typical Povidone-iodine solution the blinking effect occurs quickly and does not last very long, viscous Povidone-iodine solution may provide the practitioner with additional time to observe the blinking effect, thereby facilitating insertion of the catheter into the urethral opening.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

1. A method of inserting a catheter into a urethral opening of a female patient, the method comprising:
locating the urethral opening;
placing an identifiable marker on and/or near the urethral opening;
applying a base coat on and/or near the urethral opening to provide a background having a first color, wherein the identifiable marker has a second color that is visible relative to the first color; and
while the identifiable marker is positioned on and/or near the urethral opening, inserting the catheter into the urethral opening at least in part based on a position of the identifiable marker.

2. (canceled)

3. The method of claim 1 wherein the identifiable marker is adhered to the base coat.

4. The method of claim 1 wherein the base coat includes one or more sterilizing agents.

5-7. (canceled)

8. The method of claim 1 wherein the identifiable marker includes an adhesive sheet defining an opening that is sized and configured to approximate the urethral opening.

9. The method of claim 1 wherein the identifiable marker includes a granular material.

10. The method of claim 1 wherein the identifiable marker includes gel material.

11. (canceled)

12. The method of claim 1, further comprising inserting a positional blocker into a vaginal canal of the female patient.

13. The method of claim 1 wherein inserting the catheter into the urethral opening includes moving the catheter along a guide on a glove of a user.

14. The method of claim 13 wherein the guide is attached to a finger of the glove.

15. The method of claim 14 wherein a portion of a finger of the glove is moveable relative to and away from the guide, and a portion of the finger is fixedly attached to the guide.

16. A glove configured for insertion of a catheter into a urethral opening of a female patient, the glove comprising:
a shell defining a shape of the glove including a palm and multiple fingers, the shell including a flexible material; and
a guide extending along a finger of the multiple fingers of the shell, at least a portion of the guide being attached to or integrated with the finger, material of the guide defining a channel extending from a proximal end toward a distal end of the guide and being sized and configured to movably accept a catheter therein.

17. The glove of claim 16 wherein a portion of the guide is detached from the finger, such that a portion of the finger is moveable relative to and away from the guide.

18. The glove of claim 17 wherein the guide includes a material configured to reduce friction between the channel of the guide and the catheter.

19. The glove of claim 17 wherein the finger is an index finger of the shell.

20. The glove of claim 16, further comprising a positional blocker removably attached to a middle finger of the multiple fingers of the shell, the positional blocker being sized and configured to be inserted into a patient's vaginal canal.

21. The glove of claim 20 wherein the positional blocker is oriented approximately perpendicular relative to the middle finger of the shell.

22. A sterilizing solution for sterilizing catheterization site and identifying a urethral opening of a female patient, the sterilizing solution comprising:
an iodine solution; and
a reactive material mixed configured to react with one or more of urine or urethral mucus, the reaction producing a color change in a reacted portion of the reactive material.

23. The sterilizing solution of claim 22 wherein the reactive material is configured to change color in response to a reaction with ketones in urine.

24. The sterilizing solution of claim 23 wherein the reaction produces a purple color.

25. The sterilizing solution of claim 22 wherein the reactive material is configured to change color in response to a reaction with creatinine in urine.

26. The sterilizing solution of claim 25 wherein the reaction produces a purple color.

27. The sterilizing solution of claim 22 wherein the reactive material is configured to change color in response to a reaction with urinary glucose in urine.

28. The sterilizing solution of claim 27 wherein the reaction produces a brick-red colored precipitate.

29. The sterilizing solution of claim 22 wherein the reactive material includes sodium nitroprusside.

30. The sterilizing solution of claim 22 wherein the reactive material includes alkaline picrates.

31. The sterilizing solution of claim 22 wherein the reactive material includes copper ions.

32. The sterilizing solution of claim 22 wherein the reactive material includes glucose oxidase enzyme.

33. The sterilizing solution of claim 32 wherein the reaction produces a brown color.

34. A method of inserting a catheter into a urethral opening of a female patient, the method comprising:
locating the urethral opening;
placing an identifiable marker on and/or near the urethral opening, wherein the identifiable marker includes one or more materials reactive with one or more bodily fluids at and/or near the urethral opening; and
while the identifiable marker is positioned on and/or near the urethral opening, inserting the catheter into the urethral opening at least in part based on a position of the identifiable marker.

35. The method of claim 34 wherein reaction of the one or more materials of the identifiable marker with the one or more bodily fluids at and/or near the urethral opening changes a color of the identifiable marker.

36. The method of claim 34 wherein reaction of the one or more materials of the identifiable marker with the one or more bodily fluids at and/or near the urethral opening produces chemiluminescence of the identifiable marker.

37. The method of claim 34 wherein the identifiable marker includes a granular material.

38. The method of claim 34 wherein the one or more materials are included in a sterilizing solution.

39. A method of inserting a catheter into a urethral opening of a female patient, the method comprising:
locating the urethral opening;
detaching an identifiable marker from a glove by attaching the identifiable marker on and/or near the urethral opening and moving the glove away from the urethral opening;
placing an identifiable marker on and/or near the urethral opening; and
while the identifiable marker is positioned on and/or near the urethral opening, inserting the catheter into the urethral opening at least in part based on a position of the identifiable marker.

40. The method of claim 39 further comprising inserting a positional blocker into a vaginal canal of the female patient.

41. The method of claim 39 wherein inserting the catheter into the urethral opening includes moving the catheter along a guide on the glove of a user.

42. The method of claim 41 wherein the guide is attached to a finger of the glove.

43. The method of claim 42 wherein a portion of a finger of the glove is moveable relative to and away from the guide, and a portion of the finger is fixedly attached to the guide.

44. A method of inserting a catheter into a urethral opening of a female patient, the method comprising:
locating the urethral opening;
placing an identifiable marker on and/or near the urethral opening; and
while the identifiable marker is positioned on and/or near the urethral opening, at least in part based on a position of the identifiable marker, moving the catheter along a guide on a glove of a user to insert the catheter into the urethral opening.

45. The method of claim 44 wherein the guide is attached to a finger of the glove.

46. The method of claim 45 wherein a portion of a finger of the glove is moveable relative to and away from the guide, and a portion of the finger is fixedly attached to the guide.
